(19) United States
(12) Patent Application Publication
(10) Pub. No.: US 2015/0232893 A1
(43) Pub. Date: Aug. 20, 2015

(54) SYSTEM AND PROCESS FOR SEPARATING PURE CHEMICALS FROM BIOMASS EXTRACT

(71) Applicant: API Intellectual Property Holdings, LLC, Atlanta, GA (US)

(72) Inventors: Vesa PYLKKANEN, Atlanta, GA (US); Theodora RETSINA, Atlanta, GA (US)

(21) Appl. No.: 14/704,362

(22) Filed: May 5, 2015

Related U.S. Application Data
(62) Division of application No. 13/026,273, filed on Feb. 13, 2011.
Application No. 13/026,273 is a continuation of application No. PCT/US2010/033662, filed on May 5, 2010.
(60) Provisional application No. 61/175,588, filed on May 5, 2009.

(51) Int. Cl. C12P 7/54; C12P 7/04

(52) U.S. Cl. CPC C12P 7/54; C12P 7/04; C12P 2201/00

(57) ABSTRACT

A system or plant and method for the production of pure alcohol, acetic acid or its derivatives from the extract containing hemicelluloses filtered after extraction of woody biomass or directly extracted from woody biomass. The process can be integrated with the host plant process to minimize the effect of loss of heat value from the extracted hemicelluloses and reduce the loading to the effluent plant.

### PRIORITY DATA

This patent application is a divisional patent application of U.S. patent application Ser. No. 13/026,273 filed Feb. 13, 2011, which is a continuation application of PCT/US2010/033662 filed May 5, 2010, which claims priority to U.S. Provisional Patent App. No. 61/175,588 filed May 5, 2009, each of which is hereby incorporated by reference herein.

### FIELD OF THE INVENTION

This invention relates, in general, to the post treatment of wood extracts from forest products plants. This treatment specifically converts and separates the soluble fraction of extracted woody material to industrial grade alcohol, alkaline acetate and water.

### BACKGROUND AND SUMMARY

Forest products industry effluents contain dissolved or mechanically separated wood extract components. The major wood components are lignin, hemicelluloses and cellulose. The current pulping processes preferably separate the lignin with some loss of hemicelluloses. Dissolved lignin and hemicelluloses are burned for process energy and chemical recovery in the most pulping processes. Some or all dissolved wood components from the processes end up in the effluent treatment plant. The recovery, separation, and upgrade of the degraded hemicelluloses into chemicals and derivatives are not practiced. Most common treatment consists of activated sludge wastewater treatment from which the sludge is land filled or burned.

Specifically, the steam explosion process dissolves predominantly hemicelluloses in temperatures above 160 degrees C. Hemicelluloses removed in this process is termed “extract”. The wood chips are released through a pressure reducing valve, commonly termed “blow valve” and are used in the production of medium and hard density board. A concentration of the extract through evaporation is energy intensive, although it is currently practiced to produce molasses.

Previous research indicates that ethanol, acetic acid and their byproducts can be derived from the wood extract. Especially, predominantly hardwood, produces an extract rich in acetic acid and sugars as taught by Amidon et al. in (U.S. Patent Application No. 2007/0079944 A1, Apr. 12, 2007).

Reverse osmosis membranes achieve only 40% rejection of acetic acid according to Perry's Chemical Engineering Handbook (6th ed. p. 17-26). However, 98% rejection of sodium acetate was reported by the same source.

Bartels et al. (U.S. Pat. No. 5,028,336, Jul. 2, 1991) discloses alkalizing water-soluble organic acids and removing them by nanofiltration to reduce aqueous effluent dissolved organic solids. No attempts to purify the retentate were reported.

Nothing in the prior art teaches the process to convert acetyl groups to acetic acid in the hydrolysate, evaporate and recover pure alkaline acetate using reverse osmosis membrane. The present application discloses, amongst other things, a process wherein the hemicelluloses in the wood extract can be converted to chemical products in an energy efficient process.

The present disclosure relates to, inter alia, a process for the production of alcohol and acetic acid derivatives from wood extract. Treatment of hemicelluloses in the extract through hydrolysis, evaporation, reverse osmosis, fermentation and distillation steps is used to recover and concentrate purified water, alcohol and acetate products. The process may be integrated with the host plant to reuse water and minimize process energy and water consumption.

### BRIEF DESCRIPTION OF THE DRAWING

A more complete understanding of the present invention may be obtained by reference to the following detailed description when read in conjunction with the accompanying drawing.

FIG. 1 illustrates a typical general arrangement of the unit/system/plant operations for wood extract from a steam explosion process. Other wood extract streams are possible. It is a flow diagram example of the invention process. Process steps may be in other sequences and steps may be omitted.

### DETAILED DESCRIPTION OF THE INVENTION

This description will enable one skilled in the art to make and use the invention, and it describes several embodiments, adaptations, variations, alternatives, and uses of the invention. These and other embodiments, features, and advantages of the present invention will become more apparent to those skilled in the art when taken with reference to the following detailed description of the invention in conjunction with any accompanying drawing.

As used in this specification and the appended claims, the singular forms “a,” “an,” and “the” include plural referents unless the context clearly indicates otherwise. Unless defined otherwise, all technical and scientific terms used herein have the same meaning as is commonly understood by one of ordinary skill in the art to which this invention belongs. All composition numbers and ranges based on percentages are weight percentages, unless indicated otherwise. All ranges of numbers or conditions are meant to encompass any specific value contained within the range, rounded to any suitable decimal point.

Unless otherwise indicated, all numbers expressing reaction conditions, stoichiometries, concentrations of components, and so forth used in the specification and claims are to be understood as being modified in all instances by the term “about.” Accordingly, unless indicated to the contrary, the numerical parameters set forth in the following specification and attached claims are approximations that may vary depending at least upon a specific analytical technique.

The term “comprising,” which is synonymous with “including,” “containing,” or “characterized by” is inclusive or open-ended and does not exclude additional, unrecited elements or method steps. “Comprising” is a term of art used in claim language which means that the named claim elements are essential, but other claim elements may be added and still form a construct within the scope of the claim.

As used herein, the phase “consisting of” excludes any element, step, or ingredient not specified in the claim. When the phrase “consists of” (or variations thereof) appears in a clause of the body of a claim, rather than immediately following the preamble, it limits only the element set forth in that clause; other elements are not excluded from the claim as a whole. As used herein, the phase “consisting essentially of” limits the scope of a claim to the specified elements or method steps, plus those that do not materially affect the basis and novel characteristic(s) of the claimed subject matter.

With respect to the terms “comprising,” “consisting of,” and “consisting essentially of,” where one of these three terms is used herein, the presently disclosed and claimed subject matter may include the use of either of the other two terms. Thus in some embodiments not otherwise explicitly recited, any instance of “comprising” may be replaced by “consisting of” or, alternatively, by “consisting essentially of.”

This disclosure is of a system, plant for production and a method. The disclosure below is directed primarily to methods of carrying out the invention, but the methods also encompass a system or plant for carrying out the method.

Wood chips are charged in a batch or continuous reactor vessel, commonly termed “digester” together with steam or hot water and heated to a pressure of 5 to 30 atmospheres to treat the wood chips. In some digesters extract from the wood is removed during this treatment process. The treated wood chips are drained or blown through a valve commonly termed “blow valve” then washed with water to recover the majority of dissolved wood components into the wash filtrate; alternatively dilute wash filtrate may be used in lieu of water. The extract and the wash filtrate are collectively termed “wood extract”. The remaining wood chips are subjected to a manufacturing process, where the wood chips are converted to the final product.

The wood extract contain dissolved xylan, glucan, mannan, arbinan, galactan and acetyl groups in oligomers of hemicelluloses as well as lignin. The wood extract has low organic solids concentration of 0.1% to 12% or more. The majority of water must be removed before an economic treatment of hemicelluloses is possible.

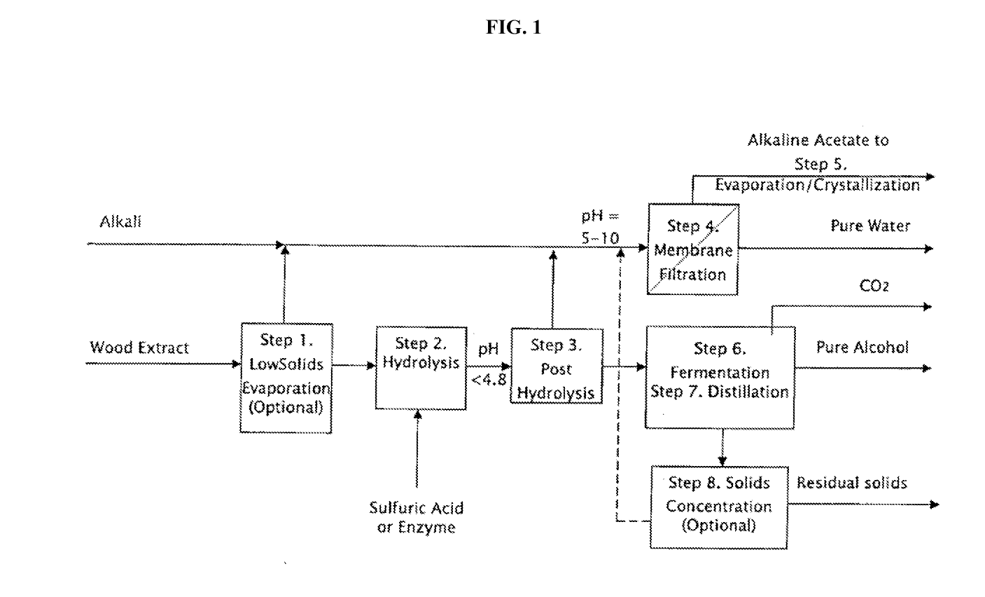

A possible first step of the process is low solids evaporation. FIG. 1 Step 1. The wood extract is concentrated preferably by evaporation, preferably using mechanical vapor recompression evaporation, to a concentration of 1% to 25% or more. If the wood extract initial concentration is over approximately 5%, this step may be omitted. When the pH is below the acetic acid dissociation point of pH 4.8, some acetic acid is split to the evaporator condensate. Under the appropriate economic criteria, this first step could be done with steam evaporation.

A second step of the process is hydrolysis. FIG. 1 Step 2. A mineral acid, preferably sulfuric acid, or enzymes is used to hydrolyze the sugars in the concentrated wood extract from the low solids evaporation step 1. Oligomer hemicelluloses are converted into monomer sugars and acetyl groups are released. The pH of the hydrolyzate from hydrolysis is controlled to maintain acetic acid in unassociated form.

A third step of the process is post hydrolysis evaporation. FIG. 1 Step 3. Hydrolyzate from step 2 is concentrated by evaporation, preferably using mechanical vapor recompression evaporation, up to 25% solids. More of the remaining acetic acid and water is evaporated in this step. Under the appropriate economic criteria, this third step could be done with steam evaporation.

A fourth step of the process is membrane filtration. FIG. 1 Step 4. Hydroxide, carbonate or bicarbonate of sodium, potassium, calcium or magnesium is added to evaporation condensates from steps 1 and 3 to convert acetic acid in the condensates to acetate. The pH of the solution should be such that nearly all acetate ions are associated, but preferably between pH 5 and 10. Acetate associated with such element produces a membrane impermeable acetate salt that is filterable in a membrane, preferably reverse osmosis membrane, with high efficiency. Because the combined condensate from evaporation contains very little impurities, the membrane permeate is a high degree of recovery as high quality water suitable for example as boiler feed water.

A fifth step of the process is acetate concentration. FIG. 1 Step 5. The retentate from the membrane in step 4 is concentrated by evaporation, preferably using mechanical vapor recompression evaporation, up to 50% solids. An industry standard finisher or crystallizer can be used to further concentrate to saleable form as may be required by the market.

A sixth step of the process is fermentation of wood sugars. FIG. 1 Step 6. Sugars in the concentrated hydrolyzate from step 3 post hydrolysis evaporation are fermented in continuous or batch tanks with one or more micro-organisms capable of converting five and six carbon sugars into alcohol and carbon dioxide. The majority of acetic acid, which may inhibit fermentation, was removed in the previous evaporation steps 1 and 3. Some additional acetic acid may be formed during fermentation. Nutrients and pH adjustment chemicals as well as make-up fermentative organism are added in the fermenters as and if needed. Carbon dioxide is removed from the fermenters and scrubbed with cool water for alcohol recovery and the purified gas can be further compressed and sold as industrial grade carbon dioxide. The fermentation broth, commonly termed “beer”, from the fermentation step is sent to step 7, distillation.

A seventh step of the process is distillation of alcohol. FIG. 1 Step 7. The beer from the step 6 fermentation is sent to a beer distillation column to separate the ethanol from the solids and residual sugars. Alcohol leaving as the overhead from the distillation column is recovered at approximately 50 mass-% strength. The final concentration of the alcohol product is performed in a rectifying column and drying system, preferably a molecular sieve, to obtain over 99 mass-% alcohol.

An eighth step of the process is the solids concentration from the stillage. FIG. 1 Step 8. The solids, commonly termed “stillage” from the beer distillation column bottom in step 7 can be further evaporated in an optional concentrator, preferably a mechanical vapor recompression-concentrator, to achieve zero-liquid discharge operation. If the sludge from the optional concentrator is burned, the process may become self-sufficient in its thermal energy needs. The condensate from this step is returned to the reverse osmosis feed in step 4.

It will be appreciated that a combination of all or any of the steps in considered part of this invention and steps may be omitted and still constitute an invention. In the preferred embodiment all disclosed steps are employed.

Integration of the Biorefinery with the Host Forest Products Plant

An energy integration analysis of the proposed process indicated that utilizing mechanical vapor recompression evaporators achieves the minimum need for cooling water. The heat generated in the process is absorbed into the product water stream, which can be utilized in the host forest products plant. Furthermore, the reverse osmosis water from step 4 is pure enough to be used in the boiler feed water makeup. This results in a reduction of the energy used in the water heating in the host forest products plant as well as unloading its waste water treatment plant operation.

The description of the invention and its applications as set forth herein is illustrative and is not intended to limit the scope of the invention. Variations and modifications of the embodiments disclosed herein are possible, and practical alternatives to and equivalents of the various elements of the embodiments would be understood to those of ordinary skill in the art upon study of this patent document. These and other variations and modifications of the embodiments disclosed herein may be made without departing from the scope and spirit of the invention.

Various aspects of the invention are as follows.

Aspect 1: A system for producing water, alcohol and acetic acid derivative from wood extract comprising: using staged evaporation, membrane separation, hydrolysis and fermentation to wood extract.

Aspect 2: A system according to Aspect 1, wherein the wood extract is a steam extract prior to a pulping process.

Aspect 3: A system according to Aspect 1, wherein the wood extract is a water extract prior to a pulping process.

Aspect 4: A system according to Aspect 1, wherein wood extract is a steam explosion extract prior to a board process.

Aspect 5: A system according to Aspect 1, wherein the wood extract is an extract prior to a biorefinery process.

Aspect 6: A system according to Aspect 1, wherein the wood extract is an extract prior to a biomass combustion process.

Aspect 7: A system according to Aspect 1, where the alcohol is cellulosic ethanol.

Aspect 8: A system according to Aspect 1, where the alcohol is butanol.

Aspect 9: A system according to Aspect 1, where evaporation is used to recover acetic acid formed in hydrolysis.

Aspect 10: A system according to Aspect 1, where mechanical vapor recompression evaporation is used to reduce cooling water consumption.

Aspect 11: A system according to Aspect 1, where acetic acid derivative is converted to sodium acetate.

Aspect 12: A system according to Aspect 1, where acetic acid derivative is converted to potassium acetate.

Aspect 13: A system according to Aspect 1, where acetic acid is converted to magnesium acetate.

Aspect 14: A system according to Aspect 1, where acetic acid is converted to calcium acetate.

Aspect 15: A system according to Aspect 1, where cellulosic ethanol is produced in zero liquid discharge facility.

Aspect 16: A system according to Aspect 1, where acetic acid derivative is converted back to acetic acid.

Aspect 17: A process for producing cellulosic ethanol and alkaline acetate acid by treatment of extract and wash filtrate from woody material comprising steps of: concentrating extract and wash filtrate in any or all of the steps, of mechanical vapor recompression evaporation, reverse osmosis membranes, and wherein hemicelluloses are hydrolyzed and the resulting sugars are fermented to alcohol, and acetic acid is collected as alkaline acetate

Aspect 18: A system according to Aspect 1, where the hydrolysis is performed with a mineral acid catalyst and resultant water is recycled.

Aspect 19: A system according to Aspect 1, where the hydrolysis is performed with sulfuric acid catalyst.

Aspect 20: A system according to Aspect 1, where the hydrolysis is performed with enzyme.

Aspect 21: A system according to Aspect 9, where the evaporation is performed with mechanical vapor recompression.

Aspect 22: A system to remove acetic acid from evaporator condensate comprising adding alkaline chemical to form membrane impermeable acetate salt.

Aspect 23: A system according to Aspect 22, where reverse osmosis membrane filtration is used.

Aspect 24: A system according to Aspect 22, where nanofiltration membrane is used.

Aspect 25: A system according to Aspect 22, where potassium acetate is the salt.

Aspect 26: A system according to Aspect 22, where sodium acetate is the salt.

Aspect 27: A system according to Aspect 22, where calcium acetate is the salt.

Aspect 28: A system according to Aspect 22, where magnesium acetate is the salt.

Aspect 29: A system according to Aspect 22, where the acetate salt is further concentrated by evaporation.

Aspect 30: A system according to Aspect 22, where the acetate salt is crystallized.

In this detailed description, reference has been made to multiple embodiments of the invention and non-limiting examples relating to how the invention can be understood and practiced. Other embodiments that do not provide all of the features and advantages set forth herein may be utilized, without departing from the spirit and scope of the present invention. This invention incorporates routine experimentation and optimization of the methods and systems described herein. Such modifications and variations are considered to be within the scope of the invention defined by the claims.

All publications, patents, and patent applications cited in this specification are herein incorporated by reference in their entirety as if each publication, patent, or patent application were specifically and individually put forth herein.

Where methods and steps described above indicate certain events occurring in certain order, those of ordinary skill in the art will recognize that the ordering of certain steps may be modified and that such modifications are in accordance with the variations of the invention. Additionally, certain of the steps may be performed concurrently in a parallel process when possible, as well as performed sequentially.

Therefore, to the extent there are variations of the invention, which are within the spirit of the disclosure or equivalent to the inventions found in the appended claims, it is the intent that this patent will cover those variations as well. The present invention shall only be limited by what is claimed.

What is claimed:

1. A process for producing an alcohol and an acetate from a biomass-derived extract, said process comprising:
(a) providing a liquid extract produced during cooking of biomass, wherein said liquid extract comprises hemicelluloses and lignin;
(b) if said liquid extract contains a first solids concentration of about 5 wt % or less, concentrating said liquid extract in a first evaporation stage, to produce a concentrated extract, wherein a first amount of acetic acid is evaporated and recovered from said first evaporation stage;
(c) hydrolyzing said liquid extract from step (a) or said concentrated extract from step (b) in the presence of a mineral acid or enzymes, to produce a hydrolyzate comprising monomer sugars and released acetyl groups;
(d) introducing said hydrolyzate to a second evaporation stage, operated at a pH less than 4.8, wherein a second amount of acetic acid is evaporated and recovered from said second evaporation stage;
(e) combining said first and second amounts of acetic acid with an alkali, at a pH selected from 5 to 10, to convert acetic acid to an acetate salt;
(f) filtering out said acetate salt with a membrane; and
(g) fermenting said monomer sugars to an alcohol.

2. The process of claim 1, wherein said cooking of biomass comprises steam cooking, steam explosion, or hot-water cooking

3. The process of claim 1, wherein said second evaporation stage concentrates said hydrolyzate to a second solids concentration up to about 25 wt %.

4. The process of claim 1, wherein mechanical-vapor recompression is utilized in said first evaporation stage, said second evaporation stage, or both first and second evaporation stages.

5. The process of claim 1, wherein said alkali is selected from the group consisting of sodium hydroxide, potassium hydroxide, calcium hydroxide, magnesium hydroxide, sodium carbonate, potassium carbonate, calcium carbonate, magnesium carbonate, sodium bicarbonate, potassium bicarbonate, calcium bicarbonate, and magnesium bicarbonate.

6. The process of claim 1, wherein said membrane is a reverse-osmosis membrane with a reverse-osmosis concentrate comprising said acetate salt and a reverse-osmosis permeate comprising water.

7. The process of claim 1, said process further comprising concentrating or crystallizing said acetate salt.

8. The process of claim 1, wherein said acetate salt is selected from the group consisting of sodium acetate, potassium acetate, calcium acetate, and magnesium acetate.

9. The process of claim 1, wherein said alcohol is ethanol or butanol.

10. The process of claim 1, said process further comprising distilling and drying said alcohol to produce a purified alcohol and a distillation bottoms stream.

11. The process of claim 10, said process further comprising introducing said distillation bottoms stream to a third evaporation stage, to produce residual solids.

12. The process of claim 11, wherein a liquid stream from said third evaporation stage is recycled to said membrane.

13. The process of claim 1, wherein said process is a zero-liquid-discharge process.

14. The process of claim 1, wherein said liquid extract is produced prior to, or during, a biomass process selected from the group consisting of a pulping process, a board process, and a combustion process.
